(19) United States
(12) Patent Application Publication
(10) Pub. No.: US 2014/0335123 A1
(43) Pub. Date: Nov. 13, 2014

(54) Food Composition and Uses for Diabetes

(71) Applicant: Kyoungsik Pak, King of Prussia, PA (US)

(72) Inventor: Kyoungsik Pak, King of Prussia, PA (US)

(21) Appl. No.: 14/260,494

(22) Filed: Apr. 24, 2014

Related U.S. Application Data
(60) Provisional application No. 61/815,794, filed on Apr. 25, 2013.

(51) Int. Cl. A23L 1/168; A61K 45/06; A61K 36/02; A61K 36/899; A61K 36/82

(52) U.S. Cl. CPC A23L 1/168; A61K 36/899; A61K 36/82; A61K 36/02; A61K 45/06
USPC 424/195.17; 426/619; 426/2

(57) ABSTRACT

The present invention provides a composition to promote healthy weight and reduce diabetic complications. The composition provides biologically active ingredients that are combined in a food and provide dietary fiber, trace minerals and phytonutrients, wherein the composition may be used alone or in combination with other foods in a method for improving health.

### CROSS REFERENCE TO RELATED APPLICATIONS

This application is a U.S. provisional application.

### BACKGROUND OF THE INVENTION

1. Field of the Invention

The invention relates to the preparation and use of a food for promoting healthy weight and stable serum glucose levels.

2. Background and Related Art

Diabetes (Diabetes mellitus, DM) is a metabolic disorder characterized by the handling of glucose by the body. Glucose is derived from dietary sources by processes of digestion and from biochemical processes. Glucose released from food enters the blood stream and requires the concomitant release of insulin to drive cellular uptake and utilization by the cells of the body. Glucose is an aldol (aldehyde containing sugar) and reactive with other serum components such as hemoglobin, other proteins, cells and tissues that circulate or make up tissues such as nerves and blood vessels. In particular, uncontrolled elevated blood glucose or hyperglycemia can cause both microvascular and macrovascular damage leading to retinopathy, nephropathy, neuropathy, and cardiovascular events.

The association between chronic hyperglycemia and increased risk of microvascular complications in patients with type 1 DM was demonstrated in the Diabetes Control and Complications Trial (DCCT) (The Diabetes Control and Complications Trial Research Group. N Engl J Med. Sep. 30 1993; 329(14):977-86). In that trial, intensive therapy designed to maintain normal blood glucose levels greatly reduced the development and progression of retinopathy, microalbuminuria, proteinuria, and neuropathy, as assessed over 7 years. Several landmark studies have confirmed the effectiveness of glycemic control in the management of type 2 DM. Improved glycemic control using injected insulin or the use of other agents has been shown to reduce the risk of major cardiovascular events, but may increase the risk of severe hypoglycemia.

People with diabetes also have an increased risk of having high serum cholesterol and a tendency toward obesity. Elevated cholesterol is believed to lead to cardiovascular damage by attaching to or contributing to the formation of aterial plaques.

Thus, there is a need for a convenient and safe products that limit or attenuate intervals of elevated blood sugar (hyperglycemia) and control blood sugar and blood cholesterol in individuals prone to exhibiting high blood glucose levels after eating and who may also have high blood lipid levels.

### SUMMARY OF THE INVENTION

The present invention provides embodiments intended for use by subjects to promote healthy body weight and for the improvement or prevention of symptoms and complications of diabetes comprising dietary fiber, phytonutrients and trace minerals; wherein the composition comprises an oatmeal and ground tea, one or more of a kelp and a seaweed, and optionally, comprises a cornmeal, and, optionally, cinnamon. In the first aspect, the present invention provides a food for use by a subject to reduce the complications of hyperglycemia and improve parameters measurable in the blood such as the presence of glycated proteins, for example, hemoglobin A1c. In one embodiment the food comprises an oatmeal, a tea, one or more of an edible seaweed and, optionally, a cornmeal, whereby the food, when ingested, promotes satiety and promotes gastric function while providing essential macronutrients and micronutrients. In one aspect the invention is a composition for the preparation of a food for use by a subject to promote healthy weight and reduce diabetic complications comprising an oatmeal and green tea is provided. In a second aspect, the composition of the invention comprises oatmeal and green tea in a ratio of dry weight from about 2:1 to about 100:1 and oatmeal and a seaweed in a ration of about 1:25 to about 200:1.

In a third aspect, the present invention provides a method of preparing the food for use to reduce the complications of hyperglycemia and reduce diabetic symptoms or complications.

In a fourth aspect, the present invention provides a method of using the compositions of the invention by a subject to promote healthy weight and reduce diabetic complications by orally administering a food comprising the composition of the present invention at least one time in a day and at least once in a given time period. In aspects of the present invention the food composition is ingested in order to improve a symptom of diabetes selected from a elevated postprandial blood glucose, paresthesia, and angina.

In a fifth aspect, a kit comprising packaging material, a container comprising a composition of the invention, and instructions for preparing a food according to the embodiments of the invention. In one embodiment, the composition provided is ready to eat. In another embodiment, the composition is used to prepare a food which is a beverage or is baked or cooked to produce a porridge, a cookie, a cake, or a cracker. In another embodiment, the composition is a semi-sold suitable for spreading or applying to another comestible, such as a fruit, vegetable, or cracker.

In another embodiment of the invention, is provided a composition in a package with instructions for the preparation and method using of the food prepared from the packaged ingredients to promote healthy weight and reduce the symptoms and complications of diabetes.

### DESCRIPTION OF THE DRAWING

FIG. 1 is a graph showing the change in blood glucose of a human subject after consuming a meal from a fast food restaurant (Trial 1), or consuming a meal comprising one embodiment of the present composition as described and prepared herein (Trials 2-5) as detailed in Example 1.

### DETAILED DESCRIPTION OF THE INVENTION

In order that the present invention may be more readily understood, certain abbreviations and terms are first defined. Additional definitions are set forth throughout the detailed description. Abbreviations used, include: ECGC=epigallocatechin-3-O-gallate; mcg=microgram; mg=milligram; gm=gram(s); kcal=kilocalories; BMI=body mass index.

All publications, including but not limited to patents and patent applications, cited in this specification are herein incorporated by reference as though fully set forth.

### DEFINITIONS

By “promote healthy weight” is meant that the consumption of excess food energy (e.g. as measured by kcal) is reduced in order to assist the subject in maintaining a body weight which has been shown by actuarial information comparing height and weight of individuals to morbidity and mortality data to reduce disease and extent lifespan. Healthy weight is also sometimes reported as “ideal body weight” based on such tables or a healthy BMI.

By “reduces postprandial blood sugar levels” or “improves the postprandial excursions of blood glucose” is meant that the excursions (increase from preprandial level) of serum glucose following ingestion of a meal are attenuated.

By “food” is meant any comestible.

As used herein, the term “subject” includes any human, nonhuman primate, or nonhuman animal, a tissue of an animal, e.g. a mammal, including a human. Non human animals subject to treatment include, for example, without limitation, a simian; a murine animal, a canine; livestock; sport animals (e.g. horses); and companion animals.

### Overview the Invention

The present invention is based on the applicant's knowledge of pharmacology as well as the natural properties of whole grains, tea, and edible sea vegetables such as kelp and seaweeds which contain digestible and indigestible fibers and other compounds and micronutrients. The applicant has unexpectedly found a combination of natural ingredients that when combined in the ratios described herein and used to produce a food, provide the health benefits of promoting a healthy body weight and improvement or prevention of the complications or diabetes. While not wishing to be bound by any one theory, the method of preparing and using the compositions provided by the present invention is believed to provide a functional food useful for treating or preventing symptoms and complications of diabetes while providing a food that promotes the sensation of satiety and other perceived or measurable health benefits.

### Dietary Fiber

The term “dietary fiber”, as it has become to be understood, is that fiber that consists of the remnants of edible plant materials, polysaccharides, lignin and associated substances resistant to (hydrolysis) digestion, that is, are nondigestible by the alimentary enzymes of humans. This definition identifies a macroconstituent of foods that includes cellulose, hemicellulose, lignin, gums, modified celluloses, mucilages, oligosaccharides, and pectins and associated minor substances, such as waxes, cutin, and suberin.

Based on their physical characteristics, fibers can be divided into two groups, water-soluble and water insoluble. Soluble fibers include gum, pectin, amylose. Insoluble fibers include cellulose, hemicelluloses, lignins, and psyllium husk.

Foods typically have both soluble and insoluble fibers. Many fruits and vegetables contain both soluble and insoluble fiber. For example, beans and vegetables are good sources of both types of fiber. For most foods, however, distinctions are made based on which type of fiber is predominant. Good sources of digestible fiber include citrus fruits, apples, peas, and barley. Nondigestible fiber can be found in high concentrations in whole grain foods, such as whole-grain meal or flour, nuts and seeds.

Digestible fiber is comprised of predominantly alpha-1,4 and alpha-1,6 glycosidic linkages that are cleavable by enzymes of humans and other higher animals. Beta-D-glucans, usually referred to as beta-glucans, comprise a class of non-digestible polysaccharides widely found in nature in sources such as grains, barley, yeast, bacteria, algae and mushrooms. In oats, barley and other cereal grains, they are located primarily in the endosperm cell wall. Beta D-glucans by nature comprise a beta-D-glycosidic linkage between the saccharides (sugar units) of the polymer which are not cleavable by the enzymes of the animal digestive system, although though within the digestive tract, they may be attached by the flora (microbes) inhabiting the gut. Inulin, for example, is a non-absorbable, nondigestible polysaccharide found in dahlia tubers, Jerusalem artichoke, or chicory root. Inulin was historically used by physicians to regulate blood sugar levels in diabetic patients at a dosage of between 25 and 50 grams per day.

Medical and nutritional studies show that fiber is essential to good health. Insoluble fiber provides a health benefit to the gastrointestinal tract by several mechanical and chemical properties, including preventing overeating and promoting satiety by its absorption of water and slowing gastric emptying. Once ingested, the mass of fiber increases as it becomes hydrated and swells thereby distending the stomach wall land activating receptors that transmit nerve impulses to the brain's satiety center. Insoluble fiber also modifies fat absorption, increases bile acid secretion, absorbs cholesterol and reduces endogenous cholesterol production, and promotes peristalsis to decreases transit time of waste out of the body. Research shows that fiber in the diet can help prevent cardiovascular disease, diabetes, obesity, gastrointestinal diseases, hypertension, osteoporosis, and several types of cancer.

Fiber has been shown to have other important benefits. By binding and speeding the removal of toxic substances from the body, dietary fiber prevents absorption of some toxins and carcinogens. Bile acids, cholesterol, and toxic heavy metals may all bind or associate with the fibers and be excreted in the stool. Fiber increases the bulk of the stool and absorbs water, helping to soften the stool and ease evacuation or reduce constipation. Maintaining fiber in the diet is recommended to prevent diverticulosis.

The average intake of dietary fiber in the United States is well below the recommended amount of between 25-40 grams of fiber per day. In one aspect, compositions of the invention provide from about 0.5 gm to about 5.0 gm of dietary fiber per serving.

### Grains

Whole grains or foods made from them contain all the essential parts and naturally occurring nutrients of the entire grain seed or kernel; all of the bran, germ, and endosperm. If the grain has been processed (e.g., cracked, crushed, rolled, extruded, and/or cooked), the food product should deliver approximately the same balance of nutrients found in the original grain seed.

By “oatmeal” is meant any preparation of the seeds of the genus Avana, such as Avena sativa or white oats. The naked oat seed with hull removed, known in the art as a “groat”, typically contains 2-4% by weight beta-glucan, depending upon oat variety and other factors such as growing conditions. Steel-cut oats, also known as pinhead oats and sometimes referred to as coarse or rough oatmeal, are made by passing groats through steel cutters which chop each one into three or four pieces but remove no portion of the oat seed. “Rolled oats” are made by steaming groats and flattening them with a roller. Rolled oats referred to as old fashioned, or jumbo, are made by first steaming the whole groat for a few minutes, thus partially cooking it, then passing it between rollers to flatten the individual seeds. Rolled oats referred to as “quick-cooking oats” are made by putting steel-cut oats through the same process. “Instant oats” or “instant oatmeal” is made in a similar fashion to rolled quick-cooking oats, except the oats are steamed longer and rolled more thinly. Oats can be ground in to flour which usually comes in three grades—coarse (ie steel-cut oats), medium and fine.

Whole grain oats also comprise phytonutrients including caffeic acid and ferulic acid found which was found to reduce the risk of colon cancer in animals. Avenanthramides, which are polyphenols found in oats, have been noted to have anti-inflammatory properties and may contribute to the skin soothing property of oats.

Oat beta-glucan is a soluble fiber. It is a viscous polysaccharide made up of units of the monosaccharide D-glucose. Oat beta-glucan is composed of mixed-linkage polysaccharides. This means that the bonds between the D-glucose or D-glucopyranosyl units are either beta-1, 3 linkages or beta-1, 4 linkages. This type of beta-glucan is also referred to as a mixed-linkage (1→3), (1→4)-beta-D-glucan. The (1→3)-linkages break up the uniform structure of the beta-D-glucan molecule and make it soluble and flexible.

Another source of beta-glucans is preparations of barley seeds as barley seeds can typically contain twice as much beta-glucan as groats.

By “a whole grain corn” is meant any ingredient that is contains whole corn kernels from the plant Zea mays, such as but without limitation: hominy, white cornmeal, yellow corn meal, masa, white corn flour, yellow corn flour, and the like. Yellow corn can have more than 10 times the vitamin A than other grains. Recent research shows that corn is also high in antioxidants and carotenoids that are associated with eye health, such as lutein and zeaxanthin. Corn is a gluten-free grain.

In one embodiment, the invention comprises a composition comprising from 0.1 to 100 gm of one or more whole grains selected from oats and barley and, optionally, a whole grain corn in the amount of 0.1 to 50 gm per serving.

### Tea

Although there are different types of tea: White, Green, Black, and Oolong; true “teas” all come from the tea plant Camellia sinensis. The more a tea plants leaves are allowed to oxidize, the darker the color of the leaf and the more stiff it is. Black tea is a heavily oxidized tea, in contrast to Green Tea, which is processed to limit oxidation. White tea is a unprocessed tea. There are numerous versions of green tea available. The different types of green tea are due to differing locations on where the tea is grown and differing climate in these locations, changes in how the tea is cultivated, and a how and when the tea is harvested and processed as well as other factors. Some names given to green tea from China are: Gunpowder, grown in the Zhejian Province, once processed, the tea looks like tiny pellets and Dragonwell, also produced in the Zhejian Province, the leaves tend to be flat and have a jade color. Green teas from Japan have the names Gyokuro, Bancha, Sensa, and Macha among others.

Numerous scientific articles confirm a variety of health benefits and the fact that green tea helps reduce weight. Green tea is rich in epigallocatechin-3-O-gallate (ECGC), a catechin that may has been reported to have anti-oxidant properties and reduce the incidence of cancer in populations consuming it. Green tea is also rich in other health-enhancing plant compounds, such as flavanols, flavanol glycosides, polyphenolic acids, caffeine, amino acids, cellulose, lipids, mono- and polysaccharides, proteins, chlorophylls and volatiles. Polyphenols that have been linked with providing antiviral, antibacterial, and anticarcinogenic properties, as well as stimulating the immune system. Further, it is thought that the antioxidants in green tea, especially, ECGC, prevent or block the formation of free radicals in the human body. Free radical damage is associated with deleterious effects on cells and tissues and generally regarded as the process of aging. Thus, green tea may help ameliorate the effects of aging, relieve headaches, including migraine headaches, eliminate or relieve angina pectoris, lower overall cholesterol levels, reduce the risk of heart attacks, lessen the likelihood of death from heart attack, decrease the risk of stroke, enhance immune functions, and aid digestion.

In one embodiment, 0.005-5 grams of tea are added per serving of the composition of the invention. The tea is green tea, white tea, black tea, oolong tea or a combination thereof.

### Edible Algae, Seaweed, or Kelp

Seaweeds come in three different color varieties, red, green and brown. Kelps, or sea vegetables, are large brown algae (Phaeophyceae) in the order Laminariales, such as Lessoniaceae, Laminariaceae, Alariaceae. There are about 30 different genera and at least 300 species of brown algae. Kelp is rich in essential nutrients, such as calcium, potassium and iodine. Iodine is an essential micronutrient and component of thyroid hormone. Thyroid hormone balance is essential for proper use of glucose and energy use by the body. Iodine may also prevent breast cancer in a manner which may or may not be related to its role as a component of thyronines (thyroid hormones T3 and T4).

Edible seaweeds or edible algae contain fiber in forms that may be purely carbohydrate but may also be sulfated polymers with beta-glycosidic bonds, e.g. agar and carrageenan, which are hydrophilic and gel forming. Red algae contain the sulfated polysaccharide carrageenan in the amorphous sections of their cell walls.

Among the seaweeds useful in practicing the invention are the common edible seaweeds: Arame (Eisenia bicyclis), Ascophyllum nodosum (Norwegian Kelp), Badderlocks (Alaria esculenta), Bladderwrack (Fucus vesiculosus), Carola (various species of Callophyllis), Carrageen moss (Mastocarpus stellatus), Channelled wrack (Pelvetia canaliculata), Chlorella, Cochayuyo (Durvillaea antarctica), Dulse (Palmaria palmata), Eucheuma spinosum, Euchema cottonii, Gutweed (Enteromorpha intestinalis), Gelidiella acerosa, Gracilaria edulis, Gracilaria corticate, Hijiki or Hiziki (Sargassum fusiforme), Hypnea, Irish moss (Chondrus crispus), layer (Porphyra laciniata/Porphyra umbilicalis), Limu Kala (Sargassum echinocarpum), Kombu (Saccharina japonica), Mozuku (Cladosiphon okamuranus), Nori (various species of the red alga Porphyra), Oarweed (Laminaria digitata), Laminaria longicruris also known as “Wild Atlantic Kombu,” Ogonori (Gracilaria), Sugar kelp (Saccharina latissima), Sea Grapes or green caviar (Caulerpa lentillifera), Sargassum cinetum, Sargassum vulgare, Sargassum swartzii, Sargassum myriocysum, Sea Lettuce (various species of the genus Ulva), Spiral wrack (Fucus spiralis), Spirulina (Arthrospira platensis and Arthrospira maxima), Thongweed (Himanthalia elongata), Wakame (Undaria pinnatifida), and Hirome or hitohame wakame (Undaria undarioides).

Ulva lactuca (sea lettuce) is a leafy, dark green, with a distinctive flavor which is very high in iron and protein, and high in iodine, aluminum, manganese and nickel. Like most sea vegetables, sea lettuce provides considerable amounts of dietary fiber (31%). Porphyra yezoensis or Nori, is a seaweed grown on nets and processed into sheets for sushi. Nori is one of the richest seaweeds in protein (up to 50 percent of the plant's dry weight) which also has dietary fiber and omega-3 fatty acids. Nori contains vitamins C (an antioxidant) and B12 and the compound taurine, which helps control cholesterol. Wakame contains a carotenoid, fucoxanthin, known to be an anti-oxidant and improve insulin resistance. Kelp is also rich in fucoidan, a phytochemical that acts as an anticoagulant. Generally, there is more iodine per gram of kelp than in other types of seaweed; ranging from 1200 mcg/gm to 8000 mcg/gm of dry material in kelp, and sometimes ten-fold or 100-fold less in seaweed.

In one embodiment, the composition comprises one or more edible forms of seaweed present at from 0.1 to 100 gm of desiccated plant per serving. In one aspect, the composition of the invention comprises from about 0.1 gm to about 10 gm of kelp and/or seaweed, about 0.1 gm to about 5 gm of kelp and/or seaweed, or from about 0.2 gm to about 5 gm of kelp and or seaweed. In one aspect, the seaweed ingredient is selected from one of the genus Laminaria, the genus Undaria, the genus Porphya, and the genus Ulva. In one aspect the composition comprises Undaria pinnatifida and Laminaria japonica.

### Additional Ingredients

Other than a the combination of active compounds providing the desired effect of improving or maintain the blood sugar level, reducing blood pressure, reduce the coagulation potential of the blood, reduce blood cholesterol, and promote vascular health. The inventive composition may comprise an additional compound or ingredient that exerts a pharmacological, nutritional, or any other beneficial activity such as an agent that prevents or hyperglycemia, hypercholstereremia, hyperlipidemia, or inflammation. It is to be understood that “other beneficial activity” may be one that is only perceived as such by the subject using the inventive compositions.

An example of an additional active ingredient is an additional vitamin or precursor, such as but without limitation: a carotenoid or retinoid (vitamin A); or a tocopherol (vitamin E); a B vitamin such as vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid), vitamin B12 (various cobalamins; commonly cyanocobalamin in vitamin supplements); an extract of a herbaceous plant or portion thereof, such as cinnamon bark or ginseng root; or a steroidal and nonsteroidal anti-inflammatory agent which may be derived from a natural source or synthetic, or other materials such as red chili pepper, parsley, aloe vera, natural or synthetic vinegar, chamomile, alpha-bisabolol, cola nitida extract, green tea extract, tea tree oil, licorice extract, allantoin, caffeine or other xanthines, glycyrrhizic acid and its derivatives.

The composition of the invention may comprise an amount of an agent that has the property of reducing fasting blood glucose levels, for example, cinnamon (Davis and Yokayama, J Med Food. 2011 September; 14(9):884-9). True cinnamon is a spice derived from a tree Cinnamomum zeylanicum. In some regions, such as the United States, cinnamon preparations may derive from the Cinnamomum aromaticum (Cinnamon cassia) and contain significant amounts of coumarin, an anticoagulant. Ingesting large and frequent amounts of coumarin by sensitive individuals may pose health risks. In an embodiment of the invention, the composition provides a serving of cinnamon from about 0.05 gm to about 5.0 gm, from about 0.10 gm to about 5.0 gm, from about 0.5 gm to about 5.0 gm, from about 0.3 gm to about 3.0 gm, or about 0.01 gm to about 3.0 gm by dry weight of cinnamon.

Additional active ingredients provided in an embodiment of the invention may include additional sources of protein or individual amino acids. In one embodiment, the composition includes a polymer, polypeptide, or protein complex which is selected from collagen, glucosamine, glucosamine and chondroitin, gelatin, lysine, tryptophan, leucine, isoleucine, histadine, an essential amino acid, or a nonessential amino acid, or any mixture thereof.

### Methods of Making the Invention

In aspects of the present invention, the food of the invention to be consumed for the beneficial health effect can be produced by any suitable process. A process of the present invention includes co-mixing the ingredients, which may be by mixing with water, and thereafter cooking them for a suitable period of time produce a food. The amount of time necessary to cook the ingredients depends on the type and amount of ingredients. Typically, for steel cut oats formulas, the cooking will take place for from about 30 to about 40 minutes and more typically about 30 minutes at about 180 degree F. Other types of oats, such as instant or rolled or flaked oats may also be used and require less cooking time to soften and produce a palatable state. If another whole grain product, such as barley is used in addition to or in place of a oatmeal, the cooking time and amount of water added per unit weight or per unit volume of the grain may be adjusted accordingly.

Generally, the composition of the invention will be produced by admixture of the individual ingredients, that is, by admixing one of a oatmeal, a tea, and, optionally, a whole grain corn ingredient and/or one or more of a seaweed. The admixed ingredients can be used alone or in combination with water or other ingredients to prepare a food of the invention. The preparation of a food of the invention may, optionally, require heating or cooking the composition until the desired texture is achieved. The individual ingredients that comprise the composition will generally be in the range given in Table 1 below.

TABLE 1

| | Minimum Dry Weight | Maximum Dry Weight |
|---|---|---|
| Ingredient | per Serving (grams) | per Serving (grams) |
| Oatmeal | 0.1 | 100 |
| Tea | 0.01 | 10 |
| Kelp | 0.1 | 100 |
| Seaweed | 0.1 | 100 |
| Cornmeal | 0.1 | 50 |
| Cinnamon | 0.05 | 5.0 |

The invention also provides for a formulation of admixed ingredients in dry or otherwise shelf stable form in proportions whereby a volume of the mixture is consumed as an individual serving after admixing with a potable liquid and, optionally, cooking, blending, baking and/or freezing the mixture. For the preparation of a bulk product, the mixture will contain the ingredients in a ration one to the other. In the formulation of the product, the ratio of oatmeal to tea by dry weight is from about 1:100 to about 10,000:1; from about 2:1 to about 100:1; from about 10:1 to about 1000:1, or from about 4:1 to about 80:1. In another embodiment of the composition which contains oatmeal, the ratio of a seaweed to the tea by weight is from about 100:1 to about 1:10,000; from about 2:1 to about 100:1; from about 10:1 to about 1000:1, or from about 5:1 to about 1:1. In another aspect, a formulation of dry ingredients comprising an oatmeal, a tea, a kelp and a seaweed is provided wherein the ratio of oatmeal to tea is from about 2:1 to about 100:1, the ratio of oatmeal to kelp is from 10:1 to about 60:1, and the ratio of oatmeal to seaweed from about 10:1 to about 30:1. In another aspect, a formulation of dry ingredients comprising an oatmeal, a tea, a kelp and a seaweed is provided wherein the ratio of oatmeal to tea is from about 50:1 to about 76:1, the ratio of oatmeal to kelp is from 10:1 to about 60:1, and the ratio of oatmeal to seaweed and/or kelp from about 10:1 to about 30:1. In one embodiment, the product contains a ratio of oatmeal: tea: seaweed and/or kelp is about 56:1:1 or is 56:1:3. When cinnamon is present as an active ingredient the ratio of oatmeal to cinnamon by dry weight is about 1:10 to about 1,000:1; from about 1:1 to about 100:1; from about 10:1 to about 2000:1, or from about 4:1 to about 100:1.

The inventive compositions, while maintaining the ratio of active ingredients as described herein, can be mixed or formulated with a carrier to form of a liquid, a gel suspension, (a semi-solid carrier), a paste; be desiccated and, optionally, milled to form a powder, such as a flour; or be suspended, dissolved; emulsified; or encapsulated into a variety of physical forms for convenience. The manufacture of shelf stable whole grain products, such as cereals, cookies, or crackers is well known in the art. The preparation of a partially hydrated or emulsified semi-solid to used as a paste or spread is also well known in the art and may require the addition of adjunctive compounds or additives such as a chelating agent, an emulsifying agent, or a preservative or anti-oxidant.

By “chelating agent” is meant a multifunctionalized compound capable of forming coordination complex with, e.g. a multivalent metal ion. Chelating agents are optionally added to the compositions of the present invention so as to enhance the preservative or preservative system and stability of the preparation. Preferred chelating agents are mild agents, such as, for example; ethylenediamine based chelaters, ethylenediamine tetraacetic acid and (EDTA, DPTA), succininc acid, citric acid and derivatives, polyhistidine, polylysine or any combination thereof.

“Emulsifiers” as used herein promote the formation and stabilization of an emulsion. Natural emulsifying agents may be derived from either animal or vegetable sources. Those from animal sources include gelatin, egg yolk, casein, animal fat, or cholesterol. Those from vegetable sources include carageenan, acacia, tragacanth, chondrus, or pectin. “An anti-oxidant agent” as used herein refers to a substance that inhibits oxidation of a substance as defined by either the loss of electrons or increase in oxidation state of a molecule or functional group. One source of oxidation is the reaction of oxygen radicals or peroxides under physiological conditions. Certain vitamins and exhibit chemical structures which make them scavengers of electrons present in oxygen radicals or be capable of reducing peroxides, among these are vitamins C (ascorbate), vitamin B3 (niacinamide and its derivatives), and vitamn E (alpha-, beta-, and gamma-tocopherols, tocopherol sorbate, tocopherol acetate, other esters of tocopherol). Phenols, such as butylated hydroxy benzoic acids and their salts, 6-hydroxy-2,5,7,8-tetramethylchroman-2-carboxylic acid (commercially available under the tradename TROLOX®), gallic acid and its alkyl esters, especially propyl gallate are known anti-oxidants. Uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcuminoids, lysine, methionine, proline, superoxide dismutase, silymarin, tea extracts, grape skin or grape seed extracts, melanin, and rosemary extracts are generally regarded as anti-oxidants.

In addition, a natural or artificial sweetner may be added. The sweetner should be one which does not contribute to excess caloric energy, or does not contribute to the symptoms or complications of diabetes. Examples of a useful sweetner are stevia plant or products with sweetner produced from stevia plants, and the like. Other examples include sucralose and aspartame.

### Methods of Using the Invention

The concentration of glucose in the human bloodstream in a resting or fasting state should be controlled within a relatively tight range (60-120 milligrams per deciliter of blood) to maintain normal health. Hypoglycemia, blood glucose below 60 mg per dL, may produce symptoms such as faintness, weakness, headache, confusion and personality changes. Severe hypoglycemia can progress to convulsions, coma and death. Excessive blood glucose, or hyperglycemia, causes subjects to exhibit excess urine production (polyuria), thirst, weight loss, fatigue, and, if severe or prolonged, can lead to dehydration, coma and death. As discussed hereinabove, chronic hyperglycemia causes tissue damage due to the chemical reactions between the excess glucose and proteins in cells, tissues, and organs. Hyperglycemia can also cause tissue damage due to swelling and edema. Diabetic symptoms and complications include: neuropathy, paresthesia, nephropathy, cardiomyopathy, retinopathy, cataract, cystopathy, corneal keratopathy, dermopathy, microangiopathy, myocardial infarction, macular edema, impaired neural conduction, wounds and skin damage, cardiovascular events, and vulnerability to infection. In addition, the compositions of the present invention provide ingredients having beneficial effects in terms of reducing or preventing or slowing absorption of glucose and lipids into the bloodstream including cholesterol, triglycerides, and fatty acids.

It is a further objective of the compositions of the invention to avoid hypoglycemia and provide nutritive value to the body by supplying macronutrients: protein, carbohydrate, dietary fiber and lipid; micronutrients: elements such as manganese, potassium and iodine; and phytochemicals which have a myriad of inherent health benefits, using a composition of the invention which comprises an oatmeal and a tea, and a kelp or seaweed, with, optionally, cornmeal. In an aspect of the invention, a food may be prepared from the composition that provides at least from about 1 g of dietary fiber to at least about 5 g of dietary fiber per serving and, provides from at least about 100 to at least about 2000 mcg of manganese per serving. In an aspect of the invention, a food may be prepared from the composition that provides at least from about 1 g of dietary fiber to at least about 5 g of dietary fiber per serving and, provides an amount of a epigallocatechin-3-O-gallate ECGC per serving.

The compositions of the present invention are used to prevent, ameliorate, or otherwise eliminate or reduce one or more of the following symptoms or complications of hyperglycemia or hyperlipidemia, such as but not limited to: obesity, overweight, weight gain, waist circumference, weight maintenance, caloric intake, body fat, Type 2 diabetes mellitus, HbA1c, dyslipoproteinemia, hyperlipoproteinemia, hyperlipidemia, hypercholesterolemia, hypertriglyceridemia, dyslipidemia, serum/plasma triglycerides, plasma lipids, serum/plasma cholesterol, LDL, HDL, lipoproteins, cholelithiasis (gall bladder disease, gall stones), hypertension, blood pressure, hypertonia, metabolic syndrome, syndrome X, coronary heart disease (CHD), cardiovascular disease (CVD), coronary artery disease, myocardial infarction, stroke, arteriosclerosis, atherosclerosis, angina pectoris, gastrointestinal cancer, polyps, adenoma, breast cancer, kidney cancer, skin damage, skin aging, skin damage or pre-mature aging, skin ulcers, paresthesia, diabetic retinopathy, macular edema, blurred vision, loss of vision, blindness, peripheral neuropathy, loss of vibration sense, temperature sense, proprioception, loss of reflexes, numbness, and painful paresthesias, and neuropathic osteoarthropathy (also known as a Charcot joint).

In a further aspect of the invention, the composition is used to promote the improvement of constipation or diverticulosis in a human or non-human mammal in need thereof. In one aspect the composition of the invention has a soothing effect on the skin of the subject.

In one aspect the composition of the invention improves a sign or symptom of diabetes or hyperlipidemia by a subjectively or objectively quantitated amount by about 1% to about 100%, about 10% to about 50%, about 10% to about 40%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of an embodiment, a preparation of the invention as disclosed herein improve a sign or symptom of diabetes or hyperlipidemia in an individual by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In certain embodiments, administration of a composition described herein may result an improvement in a sign or symptom of diabetes or hyperlipidemia of more than a 100% such as about 125%, about 150%, about 200% or more. A 200% improvement can also be expressed as a 2-fold improvement of one or more features or functions. The improvement of one or more features or functions include, but are not limited to, 3-fold, 5-fold, 10-fold, 15-fold, 20-fold, 25-fold, 75-fold, 100-fold or more, or any number therebetween.

### Dosage and Mode of Administration

For treatment of a subject, the effective amount of the composition may vary with the age, weight sex, severity of hyperglycemia, severity of hyperlipidemia, or other morbidity or co-morbidity present in a subject.

In general the subject, may be administered a composition of the invention once per day or as frequently as desired, such as once per week, once per month, or once every two weeks. The subject may be administered the composition twice or more times per day on consecutive or nonconsecutive days. As described, a dose or serving or meal of the composition prepared as proscribed, that is, by cooking, will represent a weight which is about the combined weight of the dry ingredients plus the weight of the volume of water used to prepare the finished food. Accordingly, a dose or serving of the food will generally be from about 10 gm to about 400 gm or more by weight.

A effective amount or serving of dry ingredients of the food will represent: from about 1.0 gm to about 100 gm of an oatmeal, from about 0.01 gm to about 10 gm of a tea, about 0.1 gm to about 10 gm of one or more of kelp and seaweed, and, optionally, about 1.0 gm to about 100 gm of cornmeal.

The food prepared using a composition of the invention may be in the form of a dry solid, such a non-milled admixture of the natural product ingredients, e.g. a cereal, a dry milled powder, such as a meal, flour, or beverage mix; or a compressed solid, such as a bar or a cracker or chip, or a prepared hydrated food ready to eat. It will be understood that the food can be used in any suitable manner known in the art as a comestible or to prepare a comestible; e.g. a beverage, a porridge, as a cold cereal, as an animal feed or feed supplement, as a beverage, or the like. In one aspect, the food prepared with a composition of the invention is further processed with other foods; such as fruits, nuts, berries, vegetables, salts, sweeteners, butters, meats, gelatin, or the like according to preference. In one aspect, the composition of the invention milled as appropriate for the preparation of a beverage is prepared by blending or grinding with vegetables, fresh fruit or berries to make a viscous fruit drink such as a smoothie.

The applicant has found that use of the composition of the invention regularly over a period of months has allowed him to reduce body weight and decrease the Hb A1c content in his blood. Therefore, the applicant recommends use of the composition of the invention in combination with dietary practices such as eating small amounts of protein such as beef, pork, chicken, fish, egg, milk or yogurt and salad with a small amount of carbohydrate containing food at lunchtime. The applicant further recommends eating small portions of foods at dinnertime and walking for 30 minutes in addition to use of the composition of the present invention.

While having described the invention in general terms, the embodiments of the invention will be further disclosed in the following examples.

### Example 1

### Preparation of a Hot Porridge

Examples of a serving of food of the composition of the present invention are described below. Using data provided by the United States Department of Agriculture (USDA), Nutrient Data Laboratory, Agricultural Research Station, USDA National Food and Nutrient Analysis Program; values of foods; the composition of the food produced is expected to provide:

### Composition A

The food was prepared by admixing 28 gm of rolled oatmeal, 0.5 gm of ground organic green tea, 5.0 gm of yellow cornmeal, 1.0 gm of dried kelp, and 1.5 gm seaweed. To the dry ingredients was added 1½ cup (355 ml) of water and the mixture heated to boiling. The temperature was maintained at about 200 degree Fahrenheit for about 20 with occasional stirring until the desired consistency and softness of the oatmeal was achieved.

TABLE 2

| | | |
|---|---|---|
| Calories | 138 | KCal |
| Fat | 2.3 | gm |
| Saturated | 0.4 | gm |
| Polyunsaturated | 0.9 | gm |
| Monounsaturated | 0.7 | gm |
| Carbohydrate | 24.6 | gm |
| Dietary Fiber | 3.6 | gm |
| Protein | 5.5 | gm |

A listing of some of the micronutrients and the RDI (Recommended Daily Intake) for each that are known to be present based on USDA compositions of the food ingredients are given in Table 3.

TABLE 3

| Nutrient | Amount | Unit | RDI | % of RDI |
|---|---|---|---|---|
| Calcium | 19.0 | mg | 1,200.0 | 2 |
| Cholesterol | 0.0 | mg | — | — |
| Copper | 0.22 | mg | 0.9 | 25 |
| Iron | 1.7 | mg | 8.0 | 21 |
| Magnesium | 68.4 | mg | 320.0 | 21 |
| Manganese | 2.0 | mg | 1.8 | 112 |
| Niacin | 0.48 | mg | 14.0 | 3 |
| Pant. Acid | 0.46 | mg | 5.0 | 9 |
| Phosphorus | 172.0 | mg | 700.0 | 25 |
| Potassium | 232.1 | mg | 4,700.0 | 5 |
| Riboflavin | 0.087 | mg | 1.1 | 8 |
| Selenium | 0.79 | mcg | 55.0 | 1 |
| Sodium | 13.0 | mg | 1,300.0 | 1 |
| Thiamin | 0.25 | mg | 1.1 | 23 |
| Zinc | 1.3 | mg | 8.0 | 17 |

### Composition B

The food was prepared by admixing 26 gm of rolled oatmeal, 0.5 gm of ground organic green tea, 1.0 gm of dried kelp, and 1.5 gm seaweed, and 0.5 gm of cinnamon. To the dry ingredients was added 1½ cup (355 ml) of water and the mixture heated to boiling. The temperature was maintained at about 200 degree Fahrenheit for about 20 with occasional stirring until the desired consistency and softness of the oatmeal was achieved.

The macronutrients provided by a serving as prepared are calculated in Table 4 and the micronutrients by serving as prepared are shown in Table 5.

TABLE 4

| | Grams | Calories | %-Cals |
|---|---|---|---|
| Calories | | 113 | |
| Fat | 1.8 | 15 | 13% |
| Saturated | 0.3 | 3 | 3% |
| Polyunsaturated | 0.6 | 5 | 5% |
| Monounsaturated | 0.5 | 5 | 4% |
| Carbohydrate | 20.4 | 82 | 73% |
| Dietary Fiber | 3.1 | | |
| Protein | 4.7 | 16 | 14% |

TABLE 5

| Nutrient | Amount | Unit | RDI | % of RDI |
|---|---|---|---|---|
| Calcium | 51.7 | mg | 1,200.0 | 4 |
| Cholesterol | 0.0 | mg | — | — |
| Copper | 0.15 | mg | 0.9 | 17 |
| Iron | 1.8 | mg | 8.0 | 22 |
| Magnesium | 66.4 | mg | 320.0 | 21 |
| Manganese | 1.6 | mg | 1.8 | 90 |
| Niacin | 0.3 | mg | 14.0 | 2 |
| Pant. Acid | 0.47 | mg | 5.0 | |
| Phosphorus | 129.9 | mg | 700.0 | |
| Potassium | 202.6 | mg | 4,700.0 | |
| Riboflavin | 0.1 | mg | 1.1 | |
| Selenium | 9.1 | mcmg | 55.0 | |
| Sodium | 239.5 | mg | 1,300.0 | |
| Thiamin | 0.17 | mg | 1.1 | |
| Zinc | 1.1 | mg | 8.0 | 14 |

In addition, the formulation may provide a significant source of iodine (U.S. Dept of Health and Human Services, National Institutes of Health, Office of Dietary Supplements: Iodine-Fact Sheet for Health Professions, accessed on the world wide web, Apr. 22, 2014).

### Example 2

### Test by a Human Subject

The following meals and foods were consumed by a 75 year old male subject who also test his blood glucose using a Reli On Micro® and Reli On Confirm/Micro® Blood Glucose Test Strips.

### Trial 1 04-10-2013

The subject went to fast food chain restaraunt to have dinner and ate a ¼ pound hamburger, small French fries and diet coke. Blood sugar levels were measured giving the following results:

| | |
|---|---|
| Post-prandial Time | Blood Glucose (mg/dl) |

| | | |
|---|---|---|
| 30 | min | 175 |
| 1 | hr | 220 |
| 1½ | hr | 181 |
| 2 | hr | 151 |
| 3 | hr | 137 |
| 4 | hr | 113 |

The subject stated, “After eating the hamburger dinner, my feeling was not good; my feet were tingling, headache caused high blood pressure. I did not like it at all.”

### Trial 2 on 11-2013

The subject used the following ingredients used to make a cooked porridge:

rolled oatmeal (28 gm), powdered green tea leaves (0.5 gm),
and for flavor was added a little amount apple, onion, garlic and ⅔ cube of chicken bouillon. The ingredients were added to 12 oz (355 ml) water and cooked for 20 min.

Blood sugar was measured with the following results:

| | |
|---|---|
| Post-prandial Time | Blood Glucose (mg/dl) |

| | | |
|---|---|---|
| 30 | min | 185 |
| 1 | hour | 156 |
| 1½ | hour | 133 |
| 2 | hour | 103 |
| 3 | hour | 95 |
| 4 | hour | 90 |

### Trial 3 04-12-2013

The subject ate a breakfast of porridge consisting of

rolled oatmeal (28 gm),
powdered green tea leaves (0.5 gm),
5 gm of cornmeal, and
for flavor was added small amount apple, carrots, ⅔ of beef bouillon cube.

The mixture was prepared as in Trial 2.

Blood sugar was measured with the following results:

| | |
|---|---|
| Post-prandial Time | Blood Glucose (mg/dl) |

| | | |
|---|---|---|
| 30 | min | 193 |
| 1 | hour | 165 |

| | |
|---|---|
| 1½ | 146 |

| | | |
|---|---|---|
| 2 | hour | 119 |
| 3 | hour | 109 |
| 4 | hour | 103 |

### Trial 4 04-13-2013

The subject prepared the following ingredients according to the method as in Trial 2:

rolled oatmeal (28 gm),
powdered green tea leaves (0.4 gm),
kelp (Laminaria japonica) 1.0 gm
sea weed (Undaria pinnatifida) (1.50 gm),
and for flavor was added onion, apple, garlic, 1 low sodium chicken bouillon cube.

Blood sugar was measured with the following results:

| | |
|---|---|
| Post-prandial Time | Blood Glucose (mg/dl) |

| | | |
|---|---|---|
| 30 | min | 168 |
| 1 | hour | 153 |

| | |
|---|---|
| 1½ | 122 |

| | | |
|---|---|---|
| 2 | hrs | 88 |
| 3 | hrs | 101 |
| 4 | hrs | 101 |

### Trial 5 on 15-2013

The subject prepared the following ingredients according to the method as in Trial 2 using 12

oz (355 ml) water
Rolled oatmeal (28 gm),
green tea leaves (0.4),
kelp (Laminaria japonica) 1.0 gm
sea weed (Undaria pinnatifida) (1.5 gm),
cornmeal (5 gm), and
for flavor, was added carrots, onion, garlic, chicken bouillon cube.

Also, the subject ate some salad with French dressing. Blood sugar was measured with the following results:

| | |
|---|---|
| Post-prandial Time | Blood Glucose (mg/dl) |

| | | |
|---|---|---|
| 30 | min | 158 |
| 1 | hour | 145 |
| 1½ | hour | 108 |
| 2 | hours | 99 |
| 3 | hours | 97 |
| 4 | hours | 104 |

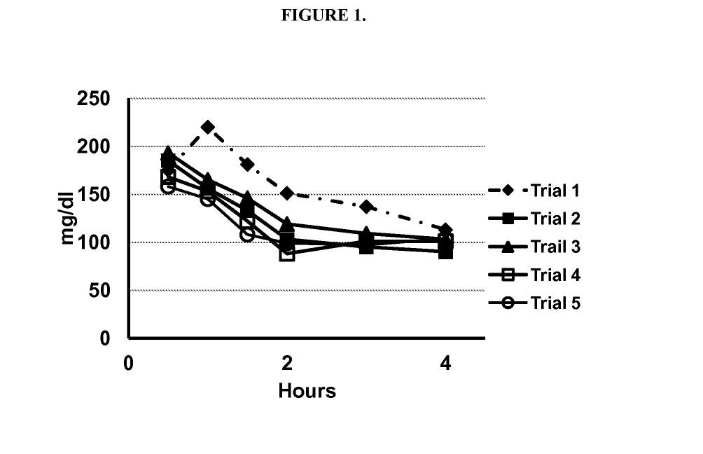

The values for blood sugar recorded during each trial were plotted together as shown in FIG. 1. The excursion of blood glucose was found to be higher for Trial 1 than for the other Trials in which compositions of the invention were ingested by the same subject.

In addition, the subject reported that after consuming variations of the porridge as described above over several months, the Hb A1c content in his blood was reduced from 6.4% on Dec. 4, 2012 to 5.8% on Apr. 16, 2013 as reported by Quest Diagnostic Labs.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term.

The terms “a,” “an,” “the” and similar referents used in the context of describing the present invention (especially in the context of the following claims) are to be construed to cover both the singular and the plural, unless otherwise indicated herein or clearly contradicted by context.

I claim:

1. A composition of a food to promote healthy body weight and for the improvement or prevention of symptoms and complications of diabetes comprising dietary fiber, phytonutrients and trace minerals; wherein the composition comprises an oatmeal and ground tea, one or more of a kelp and a seaweed, and optionally, comprises a cornmeal, and, optionally, cinnamon.

2. The composition of claim 1, wherein an effective amount of the composition in each serving of the food by dry ingredients will represent: from about 1.0 gm to about 100 gm of an oatmeal, from about 0.01 gm to about 10 gm of a tea, about 0.1 gm to about 10 gm of one or more of kelp and seaweed, optionally, about 1.0 gm to about 100 gm of cornmeal, optionally, about 0.1 gm to about 5 gm of one or more of cinnamon.

3. The composition of any of claim 2, further comprising an additional ingredient selected from the group consisting of a sweetener, an anti-inflammatory, an anti-oxidant, an omega-3 fatty acid, an emulsifying agent, and a chelating compound.

4. The composition of claim 2, wherein the oatmeal and tea is present at a ratio of from about 1:1 to about 200:1 by dry weight and the ratio of oatmeal to seaweed is from about 1:25 to about 200:1.

5. The composition of claim 2, wherein the oatmeal and cinnamon are present at a ratio at least about 10:1 and less than about 200:1.

6. The composition of claim 2, wherein the ratio of oatmeal to tea to kelp or seaweed is 56:1:3 or is 52:1:4.

7. The composition of claim 2, where in the composition provides, per serving, at least 3.0 gm of dietary fiber, provides from about 100 mcg to about 2000 mcg of manganese, and provides an amount of epigallocatechin-3-O-gallate.

8. The composition of claim 2, wherein the wherein the seaweed is one from the genuses selected from the group consisting of Laminaria, Porphyra, Ulva and Undaria.

9. The composition of claim 3, wherein the seaweed is selected from Laminaria japonica and Undaria pinnatifida (Wakame).

10. The composition of claim 3 wherein the anti-oxidant comprises one or more of vitamin C (ascorbate), vitamin B3 (niacinamide and its derivatives), vitamin E (alpha, beta, and gamma-tocopherols, tocopherol sorbate, tocopherol acetate, other esters of tocopherol; phenols, such as butylated hydroxy benzoic acids and their salts; gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione), dihydroxy fumaric acid and its salts; glycine pidolate; arginine pilolate; nordihydroguaiaretic acid; bioflavonoids; curcuminoids; lysine; methionine; proline; superoxide dismutase; silymarin; tea extracts; grape skin/seed extracts; melanin; and rosemary extracts.

11. The composition according to claim 1, wherein the physical form of the composition is selected from the group consisting of a dry flaked solid, dry solid; a dry milled powder; a semi-semisolid, and a compressed solid.

12. A food prepared from the composition of claim 11.

13. A food according to claim 12, prepared by adding a potable liquid to the composition to make a mixture and heating the mixture until the oatmeal is soft.

14. The food according to claim 12 which is a beverage prepared from the composition of claim 14 wherein the physical form of the composition is a powdered solid suitable for preparing a beverage by addition of a potable liquid.

15. The food of claim 12, which is in the form of a cookie, a cake, or a cracker.

16. The food of claim 12, wherein the food further comprises one or more of a sweetener, pH adjusting agent, a chelating agent, an emulsifying agent, an anti-oxidant agent, an anti-viral, an anti-infective, an anti-inflammatory, and a source of additional amino acids.

17. The food according to claim 16, wherein the physical form of the food is a semi-solid suitable for spreading on a fruit, a vegetable or a portion of a fruit, vegetable, or prepared food.

18. A method of using the composition of claim 1 to promote healthy body weight and improve or prevent the complications of hyperglycemia, comprising administering the composition to a subject at an amount of from about 5.0 gm to about 200 gm at least one time in a day and repeating the administration over a period of time.

19. The method of claim 18, wherein following administration of the food, the blood sugar level of the subject over four hours does not exceed 200 mg/dl.

20. A method of claim 18, wherein the complication to be improved is one or more of; obesity, overweight, weight gain, waist circumference, weight maintenance, caloric intake, body fat, Type 2 diabetes mellitus, HbA1c, dyslipoproteinemia, hyperlipoproteinemia, hyperlipidemia, hypercholesterolemia, hypertriglyceridemia, dyslipidemia, serum/plasma triglycerides, plasma lipids, serum/plasma cholesterol, LDL, HDL, lipoproteins, cholelithiasis (gall bladder disease, gall stones), hypertension, blood pressure, hypertonia, metabolic syndrome, syndrome X, coronary heart disease (CHD), cardiovascular disease (CVD), coronary artery disease, myocardial infarction, stroke, arteriosclerosis, atherosclerosis, angina pectoris, gastrointestinal cancer, polyps, adenoma, breast cancer, kidney cancer, skin damage, skin aging, skin damage or pre-mature aging, skin ulcers, diabetic retinopathy, macular edema, blurred vision, loss of vision, blindness, peripheral neuropathy, loss of vibration sense, temperature sense, proprioception, loss of reflexes, numbness, and painful paresthesia, and neuropathic osteoarthropathy (also known as a Charcot joint).
